(19) United States
(12) Patent Application Publication
(10) Pub. No.: US 2004/0235858 A1
(43) Pub. Date: Nov. 25, 2004

(54) Arylpiperazines and arylpiperidines and their use as metalloproteinase inhibiting agents

(76) Inventor: Howard Tucker, Macclesifield (GB)

Correspondence Address: ROPES & GRAY LLP, ONE INTERNATIONAL PLACE, BOSTON, MA, 02110-2624, US

(21) Appl. No.: 10/486,025

(22) PCT Filed: Aug. 8, 2002
(86) PCT No.: PCT/SE02/01438

(30) Foreign Application Priority Data
Aug. 9, 2001 (GB) 0119473.7

(51) Int. Cl. A61K 31/496; A61K 31/495

(52) U.S. Cl. 514/253.01; 514/255.02; 514/317; 544/360; 544/383; 546/194; 514/318

(57) ABSTRACT

Compounds of the formula I

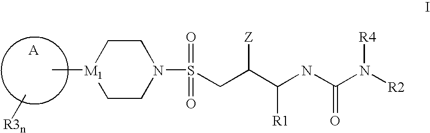

useful as metalloproteinase inhibitors, especially as inhibitors of MMP 13.

### CROSS-REFERENCE TO RELATED APPLICATIONS

This application is a national stage filing under 35 U.S.C. 371 of International Application No. PCT/SE02/01438, filed Aug. 8, 2002, which claims priority from United Kingdom Patent Application No. 0119473.7, filed Aug. 9, 2001, the specification of which is incorporated by reference herein. International Application No. PCT/SE02/01038 was published under PCT Article 21(2) in English.

### FIELD OF THE INVENTION

The present invention relates to compounds useful in the inhibition of metalloproteinases and in particular to pharmaceutical compositions comprising these, as well as their use. In particular, the compounds of this invention are inhibitors of matrix metalloproteinase 13 (MMP13), known also as collagenase 3.

Metalloproteinases are a superfamily of proteinases (enzymes) whose numbers in recent years have increased dramatically. Based on structural and functional considerations these enzymes have been classified into families and subfamilies as described in N. M. Hooper (1994) FEBS Letters 354:1-6. Examples of metalloproteinases include the matrix metalloproteinases (MMPs); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family.

Metalloproteinases are believed to be important in a plethora of physiological disease processes that involve tissue remodelling such as embryonic development, bone formation and uterine remodelling during menstruation. This is based on the ability of the metalloproteinases to cleave a broad range of matrix substrates such as collagen, proteoglycan and fibronectin. Metalloproteinases are also believed to be important in the processing, or secretion, of biological important cell mediators, such as tumour necrosis factor (TNF); and the post translational proteolysis processing, or shedding, of biologically important membrane proteins, such as the low affinity IgE receptor CD23 (for a more complete list see N. M. Hooper et al., (1997) Biochem J. 321:265-279).

Metalloproteinases have been associated with many diseases or conditions. Inhibition of the activity of one or more metalloproteinases may well be of benefit in these diseases or conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; extracellular matrix remodelling observed in cardiovascular diseases such as restenosis and atheroscelerosis; and chronic obstructive pulmonary diseases, COPD (for example, the role of MMPs such as MMP 12 is discussed in Anderson & Shinagawa, 1999, Current Opinion in Anti-inflammatory and Immunomodulatory Investigational Drugs, 1(1): 29-38).

The matrix metalloproteinases (MMPs) are a family of structurally-related zinc-containing endopeptidases which mediate the breakdown of connective tissue macro-molecules. The mammalian MMP family is composed of at least twenty enzymes, classically divided into four sub-groups based on substrate specificity and domain structure [Alexander & Werb (1991) in Hay, E. D. ed. “Cell Biology of the Extracellular Matrix”, New York, Plenum Press, 255-302; Murphy & Reynolds (1993) in Royce, P. M. & Steinman, B. eds. “Connective Tissue and its Heritable Disorders”, New York, Wiley-Liss Inc., 287-316; Birkedal-Hansen (1995) Curr. Opin. Cell Biol. 7:728-735]. The sub-groups are the collagenases (such as MMP1, MMP8, MMP13), the stromelysins (such as MMP3, MMP10, MMP11), the gelatinases (such as MMP2, MMP9) and the membrane-type MMPs (such as MMP14, MMP15, MMP16, MMP17). Enzyme activity is normally regulated in vivo by tissue inhibitors of metalloproteinases (TIMPs).

Because of their central role in re-modelling connective tissue, both as part of normal physiological growth and repair and as part of disease processes, there has been substantial interest in these proteins as targets for therapeutic intervention in a wide range of degenerative and inflammatory diseases, such as arthritis, atherosclerosis, and cancer [Whittaker et al (1999) Chem. Rev. 99:2735-2776].

A number of MMP inhibitor compounds are known and some are being developed for pharmaceutical uses [see for example the review by Beckett & Whittaker (1998) Exp. Opin. Ther. Patents, 8(3):259-282]. Different classes of compounds may have different degrees of potency and selectivity for inhibiting various MMPs. Whittaker M. et al (1999, Chem. Rev. 99:2735-2776) review a wide range of known MMP inhibitor compounds. They state that an effective MMP inhibitor requires a zinc binding group or ZBG (functional group capable of chelating the active site zinc(II) ion), at least one functional group which provides a hydrogen bond interaction with the enzyme backbone, and one or more side chains which undergo effective van der Waals interactions with the enzyme subsites. Zinc binding groups in known MMP inhibitors include hydroxamic acids (—C(O)NHOH), reverse hydroxamates (—N(OH)CHO), thiols, carboxylates and phosphonic acids.

We have discovered a new class of compounds that are inhibitors of metalloproteinases and are of particular interest in inhibiting MMP13. The compounds of this invention have beneficial potency and/or pharmacokinetic properties.

MMP13, or collagenase 3, was initially cloned from a cDNA library derived from a breast tumour [J. M. P. Freije et al. (1994) Journal of Biological Chemistry 269(24):16766-16773]. PCR-RNA analysis of RNAs from a wide range of tissues indicated that MMP13 expression was limited to breast carcinomas as it was not found in breast fibroadenomas, normal or resting mammary gland, placenta, liver, ovary, uterus, prostate or parotid gland or in breast cancer cell lines (T47-D, MCF-7 and ZR75-1). Subsequent to this observation MMP13 has been detected in transformed epidermal keratinocytes [N. Johansson et al., (1997) Cell Growth Differ. 8(2):243-250], squamous cell carcinomas [N. Johansson et al., (1997) Am. J. Pathol. 151(2):499-508] and epidermal tumours [K. Airola et al., (1997) J. Invest. Dermatol. 109(2):225-231]. These results are suggestive that MMP13 is secreted by transformed epithelial cells and may be involved in the extracellular matrix degradation and cell-matrix interaction associated with metastasis especially as observed in invasive breast cancer lesions and in malignant epithelia growth in skin carcinogenesis.

Recent published data implies that MMP13 plays a role in the turnover of other connective tissues. For instance, consistent with MMP13's substrate specificity and preference for degrading type II collagen [P. G. Mitchell et al., (1996) J. Clin. Invest. 97(3):761-768; V. Knauper et al., (1996) The Biochemical Journal 271:1544-1550], MMP 13 has been hypothesised to serve a role during primary ossification and skeletal remodelling [M. Stahle-Backdahl et al., (1997) Lab. Invest. 76(5):717-728; N. Johansson et al., (1997) Dev. Dyn. 208(3):387-397], in destructive joint diseases such as rheumatoid and osteo-arthritis [D. Wernicke et al., (1996) J. Rheumatol. 23:590-595; P. G. Mitchell et al., (1996) J. Clin. Invest. 97(3):761-768; 0. Lindy et al., (1997) Arthritis Rheum 40(8):1391-1399]; and during the aseptic loosening of hip replacements [S. Imai et al., (1998) J. Bone Joint Surg. Br. 80(4):701-710]. MMP13 has also been implicated in chronic adult periodontitis as it has been localised to the epithelium of chronically inflamed mucosa human gingival tissue [V. J. Uitto et al., (1998) Am. J. Pathol 152(6): 1489-1499] and in remodelling of the collagenous matrix in chronic wounds [M. Vaalamo et al., (1997) J. Invest. Dermatol. 109(1):96-101].

U.S. Pat. No. 6100266 and WO-99/38843 disclose compounds of the general formula

B—X—(CH₂)ₘ—(CR¹R²)ₙ—W—COY

for use in the manufacture of a medicament for the treatment or prevention of a condition associated with matrix metalloproteinases. Specifically disclosed is the compound N-{1S-[4-(4-Chlorophenyl) piperazine-1-sulfonylmethyl]-2-methylpropyl }-N-hydroxyformamide.

WO-00/12478 discloses arylpiperazines that are matrix metalloproteinase inhibitors, including compounds with an hydroxamic acid zinc binding group and compounds with a reverse hydroxamate zinc binding group.

We have now discovered compounds that are potent MMP13 inhibitors and have desirable activity profiles.

In a first aspect of the invention we now provide a compound of the formula I

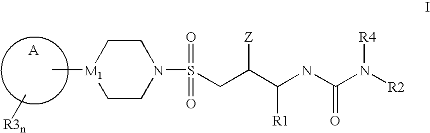

wherein

A is selected from phenyl and up to C6 heteroaryl;

n is selected from 0, 1, 2, 3;

R3 is selected from OH, NO₂, CF₃, CN, halogen, SC₁₋₄alkyl, SOC₁₋₄alkyl, SO₂C₁₋₄alkyl, C₁₋₄alkyl, C₁₋₄alkoxy, C₁₋₄alkenyl, C₁₋₄alkynyl, up to C12 cycloalkyl, up to C12 heterocycloalkyl, up to C12 aryl, up to C12 heteroaryl;

when R3 is up to C12 cycloalkyl, up to C12 heterocycloalkyl, up to C12 aryl, or up to C12 heteroaryl, R3 is optionally substituted by up to three groups independently selected from OH, NO₂, CF₃, CN, halogen, SC₁₋₄alkyl, SOC₁₋₄alkyl, SO₂C₁₋₄alkyl, C₁₋₄alkyl, C₁₋₄alkoxy, C₁₋₄alkenyl, C₁₋₄alkynyl;

M₁ is selected from N and C;

R1 is selected from H, C₁₋₆alkyl, the group Y1, and the group X1-Y1;

R2 is selected from H, C₁₋₆alkyl, the group Y2, and the group X2-Y2, or R2 together with R4 forms a five- or six-membered alkyl ring containing one or more heteroatoms independently selected from N, O, S;

R4 is selected from H and C₁₋₄alkyl, or R4 together with R2 forms a five- or six-membered alkyl ring containing one or more heteroatoms independently selected from N, O, S;

X1 and X2 are each independently C₁₋₆alkyl;

Y1 and Y2 are each independently selected from up to C10 cycloalkyl, up to C10 heterocycloalkyl, up to C10 aryl, and up to C10 heteroaryl;

Y1 and Y2 are each independently optionally substituted by up to three groups independently selected from OH, NO₂, CF₃, CN, halogen, SC₁₋₄alkyl, SOC₁₋₄alkyl, SO₂C₁₋₄alkyl, C₁₋₄alkyl, C₁₋₄alkoxy;

Z is selected from —N(OH)CHO, and —C(O)NHOH;

Any heterocycloalkyl group outlined above is an alkyl ring containing one or more heteroatoms independently selected from N, O, S;

Any heteroaryl group outlined above is an aromatic ring containing one or more heteroatoms independently selected from N, O, S;

Any alkyl group outlined above may be straight chain or branched.

Preferred compounds of the formula I are those wherein any one or more of the following apply:

A is phenyl or A is a five- or six-membered aromatic ring containing one or more heteroatoms independently selected from N, O, S; preferably A is phenyl, pyridyl, thienyl;

A is not substituted or is substituted by at least one R3 group selected from CF₃, CN, halogen (preferably fluoro or chloro), C₁₋₄alkyl;

M₁ is N;

R1 is H or Y1 or X1-Y1; preferably R1 is H or X1-Y1;

R2 is C₂₋₅alkyl or Y2 or X2-Y2; preferably R2 is C₂₋₅alkyl or Y2;

X1 is C₂₋₅alkyl; preferably X1 is C₁₋₂alkyl;

X2 is C₂₋₅alkyl; preferably X2 is C₂₋₃alkyl;

Y1 is selected from phenyl and a five- or six-membered aromatic ring containing one or more heteroatoms independently selected from N, O, S; preferably Y1 is phenyl, pyridyl, pyrimidinyl, or pyrazinyl; most preferably Y1 is phenyl;

Y1 is not substituted or is substituted by at least one group independently selected from halogen (preferably fluoro or chloro), CF₃, or MeO; preferably Y1 is not substituted or is substituted by at least one halogen group (preferably fluoro or chloro);

Y2 is selected from phenyl and a five- or six-membered aromatic ring containing one or more heteroatoms independently selected from N, O, S; preferably Y2 is phenyl, pyridyl, pyrimidinyl, or pyrazinyl; most preferably Y2 is phenyl;

Y2 is not substituted or is substituted by at least one group independently selected from halogen (preferably fluoro or chloro), CF₃, or MeO; preferably Y2 is not substituted or is substituted by at least one halogen group (preferably fluoro or chloro); Z is —N(OH)CHO.

Particularly preferred compounds of the invention are compounds of the formula IA, wherein Z is a reverse hydroxamate group:

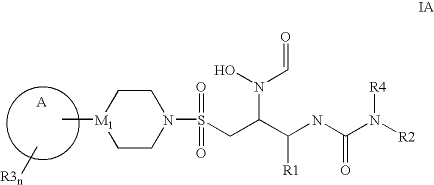

Other compounds of the invention include those wherein R2 together with R4 forms a ring selected from piperidine, piperazine, morpholine, thiomorpholine:

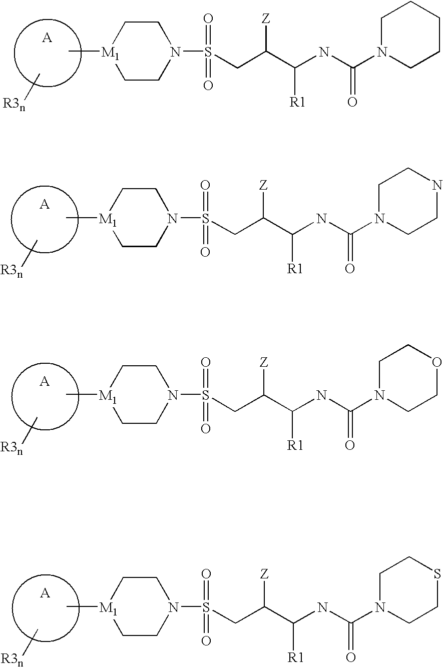

It will be appreciated that the particular substituents and number of substituents on A and/or R1 and/or R2 are selected so as to avoid sterically undesirable combinations.

Each exemplified compound represents a particular and independent aspect of the invention.

Where optically active centres exist in the compounds of formula I, we disclose all individual optically active forms and combinations of these as individual specific embodiments of the invention, as well as their corresponding racemates.

It will be appreciated that the compounds according to the invention can contain one or more asymmetrically substituted carbon atoms. The presence of one or more of these asymmetric centres (chiral centres) in a compound of formula I can give rise to stereoisomers, and in each case the invention is to be understood to extend to all such stereoisomers, including enantiomers and diastereomers, and mixtures including racemic mixtures thereof.

Where tautomers exist in the compounds of formula I, we disclose all individual tautomeric forms and combinations of these as individual specific embodiments of the invention.

As previously outlined the compounds of the invention are metalloproteinase inhibitors, in particular they are inhibitors of MMP13. Each of the above indications for the compounds of the formula I represents an independent and particular embodiment of the invention. Whilst we do not wish to be bound by theoretical considerations, the compounds of the invention are believed to show selective inhibition for any one of the above indications relative to any MMP1 inhibitory activity, by way of non-limiting example they may show 100-1000 fold selectivity over any MMP1 inhibitory activity.

The compounds of the invention may be provided as pharmaceutically acceptable salts. These include acid addition salts such as hydrochloride, hydrobromide, citrate and maleate salts and salts formed with phosphoric and sulphuric acid. In another aspect suitable salts are base salts such as an alkali metal salt for example sodium or potassium, an alkaline earth metal salt for example calcium or magnesium, or organic amine salt for example triethylamine.

They may also be provided as in vivo hydrolysable esters. These are pharmaceutically acceptable esters that hydrolyse in the human body to produce the parent compound. Such esters can be identified by administering, for example intravenously to a test animal, the compound under test and subsequently examining the test animal's body fluids. Suitable in vivo hydrolysable esters for carboxy include methoxymethyl and for hydroxy include formyl and acetyl, especially acetyl.

In order to use a compound of the formula I or a pharmaceutically acceptable salt or in vivo hydrolysable ester thereof for the therapeutic treatment (including prophylactic treatment) of mammals including humans, it is normally formulated in accordance with standard pharmaceutical practice as a pharmaceutical composition.

Therefore in another aspect the present invention provides a pharmaceutical composition which comprises a compound of the formula I or a pharmaceutically acceptable salt or an in vivo hydrolysable ester and pharmaceutically acceptable carrier.

The pharmaceutical compositions of this invention may be administered in a standard manner for the disease or condition that it is desired to treat, for example by oral, topical, parenteral, buccal, nasal, vaginal or rectal adminstration or by inhalation. For these purposes the compounds of this invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions.

In addition to the compounds of the present invention the pharmaceutical composition of this invention may also contain, or be co-administered (simultaneously or sequentially) with, one or more pharmacological agents of value in treating one or more diseases or conditions referred to hereinabove.

The pharmaceutical compositions of this invention will normally be administered to humans so that, for example, a daily dose of 0.5 to 75 mg/kg body weight (and preferably of 0.5 to 30 mg/kg body weight) is received. This daily dose may be given in divided doses as necessary, the precise amount of the compound received and the route of administration depending on the weight, age and sex of the patient being treated and on the particular disease or condition being treated according to principles known in the art.

Typically unit dosage forms will contain about 1 mg to 500 mg of a compound of this invention.

Therefore in a further aspect, the present invention provides a compound of the formula I or a pharmaceutically acceptable salt or in vivo hydrolysable ester thereof for use in a method of therapeutic treatment of the human or animal body. In particular we disclose use in the treatment of a disease or condition mediated by MMP13.

In yet a further aspect the present invention provides a method of treating a metalloproteinase mediated disease or condition which comprises administering to a warm-blooded animal a therapeutically effective amount of a compound of the formula I or a pharmaceutically acceptable salt or in vivo hydrolysable ester thereof. Metalloproteinase mediated diseases or conditions include arthritis (such as osteoarthritis), atherosclerosis, chronic obstructive pulmonary diseases (COPD).

In another aspect the present invention provides a process for preparing a compound of the formula I or a pharmaceutically acceptable salt or in vivo hydrolysable ester thereof which process comprises reacting a compound of the formula II with an appropriate compound of the formula III in which P is an appropriate protecting group to yield an alkene of the formula IV, deprotection and reaction of an amino compound of the formula V with the appropriate isocyanate or chlorocarbonylamine to yield a compound of the formula VI which is converted into a compound of the formula VII , which is a precursor to the compound of formula I.

Alternatively the alkene of the formula IV may be prepared by reaction of a compound of the formula II with an ester of the formula VIII to yield a compound of the formula IX which is reduced to a compound of the formula X and dehydrated.

The above processes are set out in the following schemes.

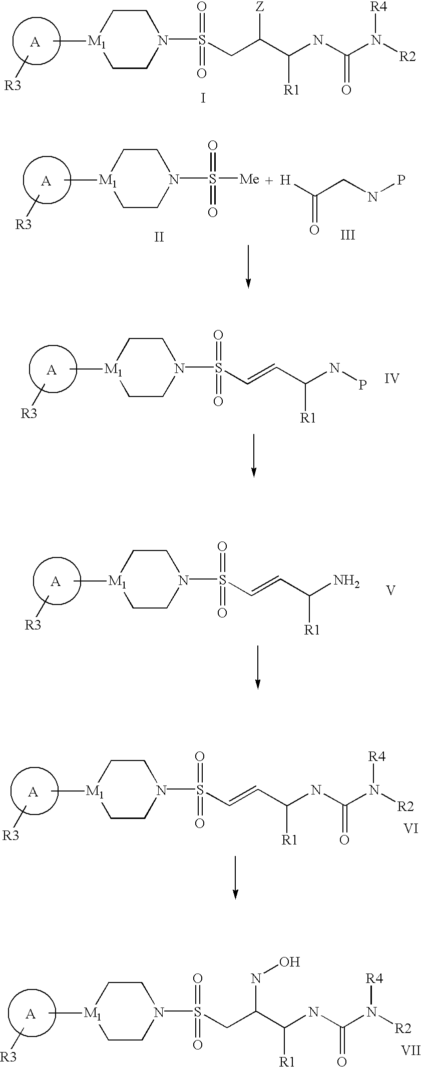

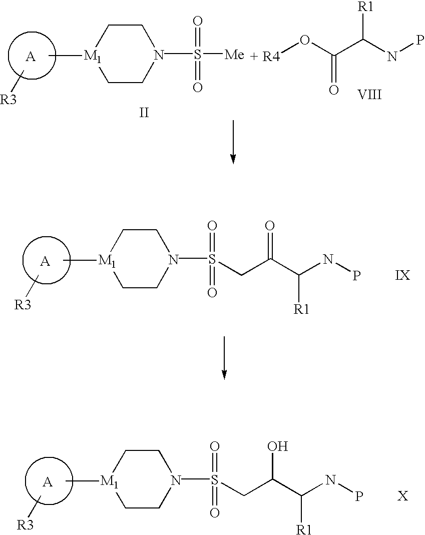

A compound of the formula IV (when M1 is N) is conveniently prepared also by reacting a compound of the formula XI with a compound of the formula XII.

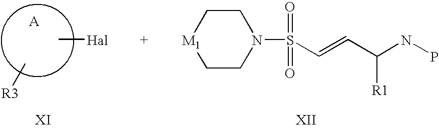

A compound of the formula XIII, where Z is CONHOH, may be prepared by reaction of a compound of the formula II with a compound of the formula XIV, in which P and P1 are appropriate protecting groups, to give a compound of the formula XV which is N-deprotected by known means to give a compound of the formula XVI which is converted into the urea of the formula XVII by reaction with the appropriate isocyanate or chlorocarbonylamine. Compounds of the formula XVII can either be deprotected and converted into the hydroxamic acids by known means or where P1 is alkyl, reacted with hydroxylamine directly.

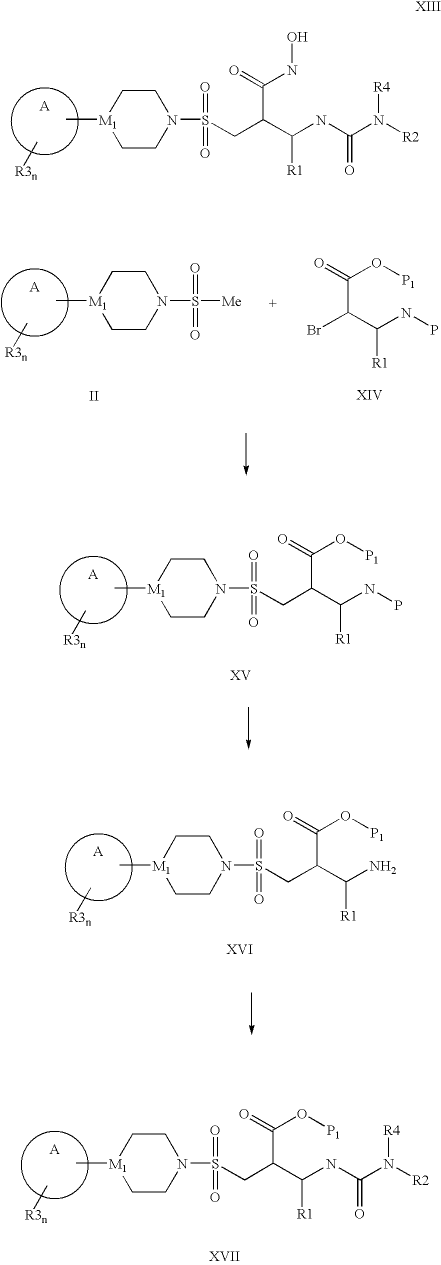

A compound of the formula XV (when M1 is N) is conveniently prepared also by reacting a compound of the formula XI with a compound of the ormula XVIII.

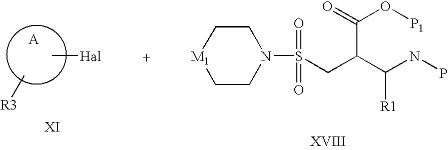

It will be appreciated that many of the relevant starting materials and intermediates are commercially available or may be made by any convenient method as described in the literature or known to the skilled chemist or described in the Examples herein. The compound of the formula II shown below is described in WO-00/12478.

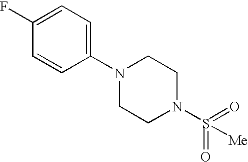

The aldehyde intermediates are commercially available and have the following CAS numbers:

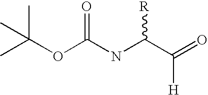

| R | Stereochemistry | CAS No. |
|---|---|---|
| H | — | 89711-08-0 |
| CH₂Ph | R | 77119-85-8 |
| CH₂Ph | S | 72155-45-4 |

The compounds of the invention may be evaluated for example in the following assays:

Isolated Enzyme Assays

Matrix Metalloproteinase Family Including for Example MMP13.

Recombinant human proMMP13 may be expressed and purified as described by Knauper et al. [V. Knauper et al., (1996) The Biochemical Journal 271:1544-1550 (1996)]. The purified enzyme can be used to monitor inhibitors of activity as follows: purified proMMP13 is activated using 1 mM amino phenyl mercuric acid (APMA), 20 hours at 21° C.; the activated MMP 13 (11.25 ng per assay) is incubated for 4-5 hours at 35° C. in assay buffer (0.1M Tris-HCl, pH 7.5 containing 0.1M NaCl, 20 mM CaCl2, 0.02 mM ZnCl and 0.05% (w/v) Brij 35 using the synthetic substrate 7-methoxycoumarin-4-yl)acetyl.Pro.Leu.Gly.Leu.N-3-(2,4-dinitrophenyl)-L-2,3-diaminopropionyl.Ala.Arg.NH₂ in the presence or absence of inhibitors. Activity is determined by measuring the fluorescence at λex 328 nm and λem 393 nm. Percent inhibition is calculated as follows: % Inhibition is equal to the [Fluorescence_{plus inhibitor}−Fluorescence_{background}] divided by the [Fluorescence_{minus inhibitor}−Fluorescence_{background}].

A similar protocol can be used for other expressed and purified pro MMPs using substrates and buffers conditions optimal for the particular MMP, for instance as described in C. Graham Knight et al., (1992) FEBS Lett. 296(3):263-266.

Adamalysin Family Including for Example TNF Convertase

The ability of the compounds to inhibit proTNFα convertase enzyme may be assessed using a partially purified, isolated enzyme assay, the enzyme being obtained from the membranes of THP-1 as described by K. M. Mohler et al., (1994) Nature 370:218-220. The purified enzyme activity and inhibition thereof is determined by incubating the partially purified enzyme in the presence or absence of test compounds using the substrate

4′,5′-Dimethoxy-fluoresceinyl

Ser.Pro.Leu.Ala.Gln.Ala.Val.Arg.Ser.Ser.Ser.Arg.Cys(4-(3-succinimid-1-yl)-fluorescein)-NH₂ in assay buffer (50 mM Tris HCl, pH 7.4 containing 0.1% (w/v) Triton X-100 and 2 mM CaCl₂), at 26° C. for 18 hours. The amount of inhibition is determined as for MMP13 except λex 490 nm and λem 530 nm were used. The substrate was synthesised as follows. The peptidic part of the substrate was assembled on Fmoc-NH-Rink-MBHA-polystyrene resin either manually or on an automated peptide synthesiser by standard methods involving the use of Fmoc-amino acids and O-benzotriazol-1-yl-N,N,N′,N′-tetramethyluronium hexafluorophosphate (HBTU) as coupling agent with at least a 4- or 5-fold excess of Fmoc-amino acid and HBTU. Ser¹ and Pro² were double-coupled. The following side chain protection strategy was employed; Ser¹(But), Gln⁵(Trityl), Arg^{8,12}(Pmc or Pbf), Ser^{9,10,11}(Trityl), Cys¹³(Trityl). Following assembly, the N-terminal Fmoc-protecting group was removed by treating the Fmoc-peptidyl-resin with 20% piperidine in DMF. The amino-peptidyl-resin so obtained was acylated by treatment for 1.5-2 hr at 70° C. with 1.5-2 equivalents of 4′,5′-dimethoxy-fluorescein-4(5)-carboxylic acid [Khanna & Ullman, (1980) Anal Biochem. 108:156-161) which had been preactivated with diisopropylcarbodiimide and 1-hydroxybenzotriazole in DMF]. The dimethoxyfluoresceinyl-peptide was then simultaneously deprotected and cleaved from the resin by treatment with trifluoroacetic acid containing 5% each of water and triethylsilane. The dimethoxyfluoresceinyl-peptide was isolated by evaporation, trituration with diethyl ether and filtration. The isolated peptide was reacted with 4-(N-maleimido)-fluorescein in DMF containing diisopropylethylamine, the product purified by RP-HPLC and finally isolated by freeze-drying from aqueous acetic acid. The product was characterised by MALDI-TOF MS and amino acid analysis.

Natural Substrates

The activity of the compounds of the invention as inhibitors of aggrecan degradation may be assayed using methods for example based on the disclosures of E. C. Arner et al., (1 998) Osteoarthritis and Cartilage 6:214-228; (1999) Journal of Biological Chemistry, 274 (10), 6594-6601 and the antibodies described therein. The potency of compounds to act as inhibitors against collagenases can be determined as described by T. Cawston and A. Barrett (1979) Anal. Biochem. 99:340-345.

Inhibition of Metalloproteinase Activity in Cell/Tissue Based Activity

Test as an Agent to Inhibit Membrane Sheddases such as TNF Convertase

The ability of the compounds of this invention to inhibit the cellular processing of TNFα production may be assessed in THP-1 cells using an ELISA to detect released TNF essentially as described K. M. Mohler et al., (1994) Nature 370:218-220. In a similar fashion the processing or shedding of other membrane molecules such as those described in N. M. Hooper et al., (1997) Biochem. J. 321:265-279 may be tested using appropriate cell lines and with suitable antibodies to detect the shed protein.

Test as an Agent to Inhibit Cell Based Invasion

The ability of the compound of this invention to inhibit the migration of cells in an invasion assay may be determined as described in A. Albini et al., (1987) Cancer Research 47:3239-3245.

Test as an Agent to Inhibit Whole Blood TNF Sheddase Activity

The ability of the compounds of this invention to inhibit TNFα production is assessed in a human whole blood assay where LPS is used to stimulate the release of TNFα. Heparinized (10Units/ml) human blood obtained from volunteers is diluted 1:5 with medium (RPMI1640+bicarbonate, penicillin, streptomycin and glutamine) and incubated (160 μl) with 20 μl of test compound (triplicates), in DMSO or appropriate vehicle, for 30 min at 37° C. in a humidified (5%CO₂/95%air) incubator, prior to addition of 20 μl LPS (E. coli. 0111:B4; final concentration 10 μg/ml). Each assay includes controls of diluted blood incubated with medium alone (6 wells/plate) or a known TNFα inhibitor as standard. The plates are then incubated for 6 hours at 37° C. (humidified incubator), centrifuged (2000 rpm for 10 min; 4° C.), plasma harvested (50-100 μl) and stored in 96 well plates at −70° C. before subsequent analysis for TNFα concentration by ELISA.

Test as an Agent to Inhibit In Vitro Cartilage Degradation

The ability of the compounds of this invention to inhibit the degradation of the aggrecan or collagen components of cartilage can be assessed essentially as described by K. M. Bottomley et al., (1997) Biochem J. 323:483-488.

Pharmacodynamic Test

To evaluate the clearance properties and bioavailability of the compounds of this invention an ex vivo pharmacodynamic test is employed which utilises the synthetic substrate assays above or alternatively HPLC or Mass spectrometric analysis. This is a generic test which can be used to estimate the clearance rate of compounds across a range of species. Animals (e.g. rats, marmosets) are dosed iv or po with a soluble formulation of compound (such as 20% w/v DMSO, 60% w/v PEG400) and at subsequent time points (e.g. 5, 15, 30, 60, 120, 240, 480, 720, 1220 mins) the blood samples are taken from an appropriate vessel into 10U heparin. Plasma fractions are obtained following centrifugation and the plasma proteins precipitated with acetonitrile (80% w/v final concentration). After 30 mins at −20° C. the plasma proteins are sedimented by centrifugation and the supernatant fraction is evaporated to dryness using a Savant speed vac. The sediment is reconstituted in assay buffer and subsequently analysed for compound content using the synthetic substrate assay. Briefly, a compound concentration-response curve is constructed for the compound undergoing evaluation. Serial dilutions of the reconstituted plasma extracts are assessed for activity and the amount of compound present in the original plasma sample is calculated using the concentration-response curve taking into account the total plasma dilution factor.

In Vivo Assessment

Test as an Anti-TNF Agent

The ability of the compounds of this invention as ex vivo TNFα inhibitors is assessed in the rat. Briefly, groups of male Wistar Alderley Park (AP) rats (180-210 g) are dosed with compound (6 rats) or drug vehicle (10 rats) by the appropriate route e.g. peroral (p.o.), intraperitoneal (i.p.), subcutaneous (s.c.). Ninety minutes later rats are sacrificed using a rising concentration of CO₂ and bled out via the posterior vena cavae into 5 Units of sodium heparin/ml blood. Blood samples are immediately placed on ice and centrifuged at 2000 rpm for 10 min at 4° C. and the harvested plasmas frozen at −20° C. for subsequent assay of their effect on TNFα production by LPS-stimulated human blood. The rat plasma samples are thawed and 175 μl of each sample are added to a set format pattern in a 96 well plate. Fifty μl of heparinized human blood is then added to each well, mixed and the plate is incubated for 30 min at 37° C. (humidified incubator). LPS (25 μl; final concentration 10 μg/ml) is added to the wells and incubation continued for a further 5.5 hours. Control wells are incubated with 25 μl of medium alone. Plates are then centrifuged for 10 min at 2000 rpm and 200 μl of the supernatants are transferred to a 96 well plate and frozen at −20° C. for subsequent analysis of TNF concentration by ELISA.

Data analysis by dedicated software calculates for each compound/dose:

Percent inhibition of TNFα=Mean TNFα (Controls)−Mean TNFα (Treated)×100 Mean TNFα (Controls)

Test as an Anti-Arthritic Agent

Activity of a compound as an anti-arthritic is tested in the collagen-induced arthritis (CIA) as defined by D. E. Trentham et al., (1977) J. Exp. Med. 146,:857. In this model acid soluble native type II collagen causes polyarthritis in rats when administered in Freunds incomplete adjuvant. Similar conditions can be used to induce arthritis in mice and primates.

Test as an Anti-Cancer Agent

Activity of a compound as an anti-cancer agent may be assessed essentially as described in I. J. Fidler (1978) Methods in Cancer Research 15:399-439, using for example the B16 cell line (described in B. Hibner et al., Abstract 283 p75 10th NCI-EORTC Symposium, Amsterdam Jun. 16-19 1998).

The invention will now be illustrated but not limited by the following Examples:

### EXAMPLE 1

Preparation of N-(4-fluorophenyl)-N′-[3-{4-fluorophenylaminocarbonyl}amino-2-{(N-formyl-N-hydroxy)aminopropane-1-sulphonyl]piperazine

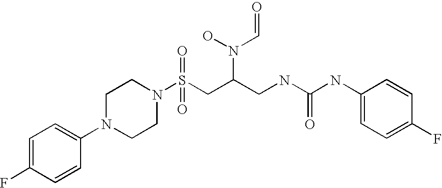

A mixture of formic acid and acetic anhydride (230 μl) [prepared by adding acetic anhydride (1 ml) to formic acid (5 ml) maintained at 0° C. and stirred at this temperature for 30 minutes) was added to a solution of N-(4-fluorophenyl)-N′-[3-{4-fluorophenylaminocarbonyl}amino-2-[N-hydroxyaminopropane-1-sulphonyl]piperazine (34 mg) in formic acid (0.5 ml) and the mixture was stirred for 14 hours. The reaction mixture was evaporated to dryness, the residue was dissloved in methanol (3 ml) and stirred at 40° C. for 3 hours. The solvent was evaporated and the residue was purified by chromatography on silica eluted initially with a mixture of ethyl acetate and isohexane (1:2 v/v) then moving to neat ethyl acetate. The gum obtained solidified on trituration with chloroform, yield 12 mg. MS (ES⁺) M+H=498.

The N-(4-fluorophenyl)-N′-[3-{4-fluorophenylaminocarbonyl}amino-2-[N-hydroxyaminopropane-1-sulphonyl]piperazine used as starting material was prepared as follows:

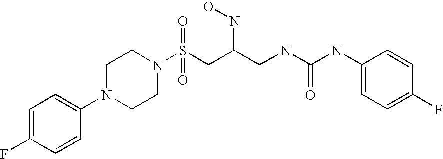

50% aqueous hydroxylamine (204 μl) was added to a solution of N-(4-fluorophenyl)-N′-[3-{4-fluorophenylaminocarbonyl}aminoprop-1-ene-1-sulphonyl]piperazine (70 mg) in THF (2 ml) and the mixture was stirred for 14 hours. The reaction mixture was filtered and the filtrate was partitioned between water and ethyl acetate (5 ml of each).

The organic layer was separated and the aqueous layer was extracted with ethyl acetate (3×5 ml). The combined ethyl acetate extracts were washed with brine, dried and evaporated to dryness. The residue was purified by chromatography on silica eluting with a solvent gradient (ethyl acetate: isohexane 1:1 to neat ethyl acetate) to give the required product as a white solid, yield 34 mg, MS (ES⁺) M+H=470.

Preparation of N-(4-fluorophenyl)-N′-[3-{4-fluorophenylaminocarbonyl}aminoprop-1-ene-1-sulphonyl]piperazine.

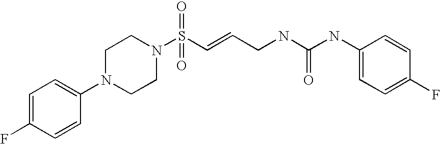

N-(4-fluorophenyl)-N′-(3-aminoprop-1-ene-1-sulphonyl)piperazine (100 mg) was added to a solution of 4-fluorophenylisocyanate (47 mg) in dichloromethane (1 ml) and the mixture was stirred for 14 hours under argon. The solid that precipitated was collected and dried. This was shown to contain N-(4-fluorophenyl)-N′-(4-fluorophenylaminocarbonyl)piperazine (˜50%) and this contaminated material was used without further purification for the next stage.

Preparation of N-(4-fluorophenyl)-N′-(3-aminoprop-1-ene-1-sulphonyl)piperazine.

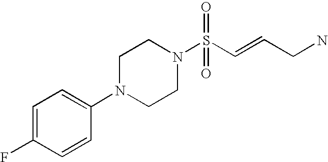

TFA (60 ml) was added to a solution of N-(4-fluorophenyl)-N′-(3-{Boc-amino}prop-1-ene-1-sulphonyl)piperazine (1.26 g) in dichloromethane (15 ml) and the reaction mixture was stirred under argon for 1 hour. The reaction mixture was evaporated to dryness and the residue was partitioned between saturated aqueous sodium bicarbonate (20 ml) and ethyl acetate (20 ml). The aqueous phase was collected and extracted with ethyl acetate (3×20 ml). The combined ethyl acetate extracts were washed with brine and dried. Evaporation to dryness gave N-(4-fluorophenyl)-N′-(3-aminoprop-1-ene-1-sulphonyl)piperazine as a pale yellow solid, yield 600 mg, MS (ES⁺) M+H=300.

Preparation of N-(4-fluorophenyl)-N′-(3-{Boc-amino}prop-1-ene-1-sulphonyl)piperazine.

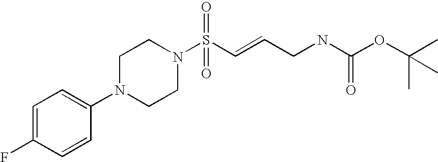

Lithium bis(trimethylsilyl)amide (13.4 ml of 1.0M solution in THF) was added dropwise to a stirred solution of N-(4-fluorophenyl)-N′-(methanesulphonyl)piperazine (1.57 g) in THF (60 ml) maintained at −15° C. at such a rate that the temperature did not exceed −10° C. The reaction mixture was stirred at −15° C. for 20 minutes and diethylchlorophosphate (0.925 ml) was added and stirring continued for 15 minutes. A solution of Boc-aminoacetaldehyde (0.98 g) in THF (15 ml) was added dropwise and the mixture was stirred at −10° C. for 2 hours. Saturated aqueous ammonium chloride was added (1 ml) and the mixture was evaporated to small volume and diluted with water. The mixture was extracted with diethyl ether (3×25 ml) and the combined ether extracts were washed with brine and dried. The solvent was removed and the residue was purified by chromatography on silica luting with a solvent gradient starting with neat isohexane and finishing with neat ethyl acetate. N-(4-fluorophenyl)-N′-(3-{Boc-amino}prop-1-ene-1-sulphonyl)piperazine was obtained, yield 1.26 g. MS (ES⁺) M+H=400.

### EXAMPLE 2

The compounds below were prepared according to the procedures described in Example 1 using the appropriate isocyanates and aldehydes.

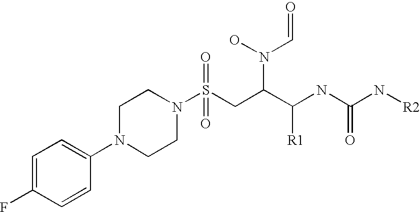

| | | Mass spectrum |
|---|---|---|
| R1 | R2 | (M + H) |
| H | Ph | 480 |
| H | isopropyl | 446 |
| H | 3-Fluoro-Ph | 498 |
| (R)-benzyl | Ph | 570 |
| (S)-benzyl | Ph | 570 |

What we claim is:

1. A compound of the formula I or a pharmaceutically acceptable salt or an in vivo hydrolysable ester thereof, 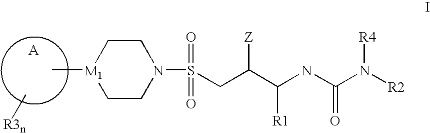
wherein
A is selected from phenyl and up to C6 heteroaryl;
n is selected from 0, 1, 2, and 3;
R3 is selected from OH, NO₂, CF₃, CN, halogen, SC₁₋₄alkyl, SOC₁₋₄alkyl, SO₂C₁₋₄alkyl, C₁₋₄alkyl, C₁₋₄alkoxy, C₁₋₄alkenyl, C₁₋₄alkynyl, up to C12 cycloalkyl, up to C12 heterocycloalkyl, up to C12 aryl, and up to C12 heteroaryl; wherein
when R3 is up to C12 cycloalkyl, up to C12 heterocycloalkyl, up to C12 aryl, or up to C12 heteroaryl, R3 is optionally substituted by up to three groups independently selected from OH, NO₂, CF₃, CN, halogen, SC₁₋₄alkyl, SOC₁₋₄alkyl, SO₂C₁₋₄alkyl, C₁₋₄alkyl, C₁₋₄alkoxy, C₁₋₄alkenyl, and C₁₋₄alkynyl;
M₁ is selected from N and C;
R1 is selected from H, C₁₋₆alkyl, the group Y1, and the group X1-Y1;
R2 is selected from H, C₁₋₆alkyl, the group Y2, and the group X2-Y2, or R2 together with R4 forms a five- or six-membered alkyl ring containing one or more heteroatoms independently selected from N, O, and S;
R4 is selected from H and C₁₋₄alkyl, or R4 together with R2 forms a five- or six-membered alkyl ring containing one or more heteroatoms independently selected from N, O, and S;
X1 and X2 are each independently C₁₋₆alkyl;
Y1 and Y2 are each independently selected from up to C10 cycloalkyl, up to C10 heterocycloalkyl, up to C10 aryl, and up to C10 heteroaryl;
Y1 and Y2 are each independently optionally substituted by up to three groups independently selected from OH, NO₂, CF₃, CN, halogen, SC₁₋₄alkyl, SOC₁₋₄alkyl, SO₂C₁₋₄alkyl, C₁₋₄alkyl, and C₁₋₄alkoxy; and
Z is selected from —N(OH)CHO and —C(O)NHOH.

2. A compound of the formula IA or a pharmaceutically acceptable salt or an in vivo hydrolysable ester thereof, 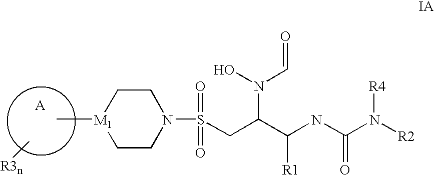
wherein
A is selected from phenyl and up to C6 heteroaryl;
n is selected from 0, 1, 2, and 3;
R3 is selected from OH, NO₂, CF₃, CN, halogen, SC₁₋₄alkyl, SOC₁₋₄alkyl, SO₂C₁₋₄alkyl, C₁₋₄alkyl, C₁₋₄alkoxy, C₁₋₄alkenyl, C₁₋₄alkynyl, up to C12 cycloalkyl, up to C12 heterocycloalkyl, up to C12 aryl, and up to C12 heteroaryl;
when R3 is up to C12 cycloalkyl, up to C12 heterocycloalkyl, up to C12 aryl, or up to C12 heteroaryl, R3 is optionally substituted by up to three groups independently selected from OH, NO₂, CF₃, CN, halogen, SC₁₋₄alkyl, SOC₁₋₄alkyl, SO₂C₁₋₄alkyl, C₁₋₄alkyl, C₁₋₄alkoxy, C₁₋₄alkenyl, and C₁₋₄alkynyl;
M₁ is selected from N and C;
R1 is selected from H, C₁₋₆alkyl, the group Y1, and the group X1-Y1;
R2 is selected from H, C₁₋₆alkyl, the group Y2, and the group X2-Y2, or R2 together with R4 forms a five- or six-membered alkyl ring containing one or more heteroatoms independently selected from N, O, and S;
R4 is selected from H and C₁₋₄alkyl, or R4 together with R2 forms a five- or six-membered alkyl ring containing one or more heteroatoms independently selected from N, O, and S;
X1 and X2 are each independently C₁₋₆alkyl;
Y1 and Y2 are each independently selected from up to C10 cycloalkyl, up to C10 heterocycloalkyl, up to C10 aryl, and up to C10 heteroaryl; and
Y1 and Y2 are each independently optionally substituted by up to three groups independently selected from OH, NO₂, CF₃, CN, halogen, SC₁₋₄alkyl, SOC₁₋₄alkyl, SO₂C₁₋₄alkyl, C₁₋₄alkyl, and C₁₋₄alkoxy.

3. A compound of claim 1 or claim 2 or a pharmaceutically acceptable salt or an in vivo hydrolysable ester thereof, wherein A is phenyl or A is a five- or six-membered aromatic ring containing one or more heteroatoms independently selected from N, O, and S.

4. A compound of claim 1 or claim 2 or a pharmaceutically acceptable salt or an in vivo hydrolysable ester thereof, wherein A is not substituted or is substituted by at least one R3 group selected from CF₃, CN, halogen, and C₁₋₄alkyl.

5. A compound of claim 1 or claim 2 or a pharmaceutically acceptable salt or an in vivo hydrolysable ester thereof, wherein M₁ is N.

6. A compound of claim 1 or claim 2 or a pharmaceutically acceptable salt or an in vivo hydrolysable ester thereof, wherein R1 is H, Y1 or X1-Y1.

7. A compound of claim 1 or claim 2 or a pharmaceutically acceptable salt or an in vivo hydrolysable ester thereof, wherein R2 is C₂₋₅alkyl, Y2, or X2-Y2.

8. A compound of claim 1 or claim 2 or a pharmaceutically acceptable salt or an in vivo hydrolysable ester thereof, wherein at least one of X1 and X2 is C₂₋₅alkyl.

9. A compound of claim 1 or claim 2 or a pharmaceutically acceptable salt or an in vivo hydrolysable ester thereof, wherein at least one of Y1 and Y2 is selected from phenyl and a five- or six-membered aromatic ring containing one or more heteroatoms independently selected from N, O, and S.

10. A compound of claim 1 or claim 2 or a pharmaceutically acceptable salt or an in vivo hydrolysable ester thereof, wherein at least one of Y1 and Y2 is not substituted or is substituted by at least one group independently selected from halogen, CF₃, or MeO.

11. A compound of claim 1 or claim 2 or a pharmaceutically acceptable salt or an in vivo hydrolysable ester thereof, wherein R2 together with R4 forms a ring selected from piperidine, piperazine, morpholine, and thiomorpholine.

12. A compound as claimed in claim 1 or a pharmaceutically acceptable salt or an in vivo hydrolysable ester thereof, wherein the compound is N-(4-fluorophenyl)-N′-[3-{4-fluorophenylaminocarbonyl}amino-2-{(N-formyl-N-hydroxy)aminopropane-1-sulphonyl]piperazine.

13. A pharmaceutical composition which comprises, a pharmaceutically acceptable carrier and a compound of claim 1 or claim 2 or a pharmaceutically acceptable salt or an in vivo hydrolysable ester thereof.

14. A method for treating a human or animal, comprising administering to the human or animal a therapeutic amount of a compound of claim 1 or claim 2 or a pharmaceutically acceptable salt or in vivo hydrolysable ester thereof.

15. A method of treating a metalloproteinase mediated disease or condition which comprises, administering to a warm-blooded animal a therapeutically effective amount a compound of claim 1 or claim 2 or a pharmaceutically acceptable salt or in vivo hydrolysable ester thereof.

16. A method of treating a metalloproteinase mediated disease or condition as claimed in claim 15, wherein the metalloproteinase is MMP13.

17. A method for treating a disease or condition mediated by one or more metalloproteinase enzymes, said method comprising administering to a patient in need thereof a therapeutically effective amount of a compound of claim 1 or claim 2 or a pharmaceutically acceptable salt or in vivo hydrolysable precursor thereof.

18. A method for treating arthritis, said method comprising administering to a patient in need thereof a therapeutically effective amount of a compound of claim 1 or claim 2 or a pharmaceutically acceptable salt or in vivo hydrolysable precursor thereof.
